(19) United States
(12) Patent Application Publication
(10) Pub. No.: US 2002/0177218 A1
(43) Pub. Date: Nov. 28, 2002

(54) Methods of detecting multiple DNA binding protein and DNA interactions in a sample, and devices, systems and kits for practicing the same

(76) Inventors: Yu Fang, Fremont, CA (US); Xiao-Yang Wang, Mountain View, CA (US); Pierre Turpin, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/113,877

(22) Filed: Mar. 29, 2002

Related U.S. Application Data
(60) Provisional application No. 60/280,658, filed on Mar. 30, 2001.
(60) Provisional application No. 60/314,330, filed on Aug. 20, 2001.

(51) Int. Cl. C12N 1/20

(52) U.S. Cl. 435/252.3

(57) ABSTRACT

Methods for detecting the presence of at least one, usually a plurality of, DNA binding proteins, e.g., transcription factors, in a sample, both qualitatively and quantitatively, are provided. In the subject methods, a substrate having one or more DNA probes immobilized on a surface thereof, one for each DNA binding protein of interest, is contacted with a sample under conditions sufficient for binding complexes of the probes and their respective DNA binding proteins to be produced. The sample may be purified with respect to one or more DNA binding proteins or be a cellular/nuclear extract. Resultant binding complexes on the surface of the substrate are then detected and related to the presence of the DNA binding protein-DAN interations of interest in the sample. Also provided are devices and systems for use in practicing the subject methods. The subject methods find use in a variety of different applications, e.g., detecting the presence of a transcription factor in a sample, the study of transcription factor profiles in response to a given stimulus, screening for therapeutic agents, and the like.

### CROSS-REFERENCE TO RELATED APPLICATIONS

Pursuant to 35 U.S.C. §119 (e), this application claims priority to the filing date of U.S. Provisional Patent Application Serial No. 60/280,658 filed Mar. 30, 2001 and U.S. Provisional Patent Application Serial No. 60/314,330 filed Aug. 20, 2001; the disclosures of which are herein incorporated by reference.

### INTRODUCTION

### FIELD OF THE INVENTION

The field of this invention is DNA binding proteins, particularly transcription factors.

### BACKGROUND OF THE INVENTION

The study of the interactions between a DNA binding protein and DNA is one of the most rapidly growing areas of molecular biology. Transcription factors, a subset of DNA binding proteins, are at the heart of the regulation and control of gene expression, replication, and recombination. Because of their important roles, inhibition and stimulation of transcription factor binding to DNA is of great interest in the discovery of potential targets for new drugs.

Several different protocols have been developed to study DNA-protein interactions, such as DNA-protein photocrosslinking, south-western blotting, in vivo reporting system and the electrophoretic mobility shift assay (EMSA). EMSA is one of the most powerful tools to study the functional relationship between a DNA binding protein and its cognate DNA site. However, the EMSA has some intrinsic disadvantages such as radioactive usage, limitation of sample numbers and long assay time. These disadvantages and the need for a high throughput format have led to the development of an enzyme-linked DNA-protein binding assay to complement the traditional EMSA.

Alternative assays to EMSA have been developed, including assays based on an ELISA protocol. For example, Benotmane et al., Anal. Biochem. (1997) 250:181-185; developed an ELISA based assay in which the binding activity of purified human helicase-like transcription factor was studied. However, this assay was not tested with cellular extracts.

Likewise, McKay et al., Anal. Biochem (1998) 1:28:34 reports an ELISA based transcription factor inhibitor screening assay in which DNA probes bound to a surface are contacted with purified transcription factor in the presence of a candidate agent. Bound transcription factor is detected chromogenically and used to derive the inhibitory activity of the test compound. Again, this assay was not tested with cell extracts as opposed to purified transcription factor and the sensitivity of this assayed compared to EMSA is not provided.

Another transcription factor ELISA type assay is reported in Gubler et al., Biotechniques (1995) 18:1011-1014. In this assay, a transcription factor is first incubated with a biotinylated ds-DNA probe and an antibody for the transcription factor. The resultant mixture is then transferred to anti-lgG coated microwells, and the presence of DNA/transcription factor/antibody complexes are detected chromogenically with AP conjugated streptavidin. There are disdvantages with this method, including the fact that reactions are performed in more than one container and the lack of specificity with respect to the detection of active v. inactive transcription factor, leading to decreased sensitivity.

Yet another transcription factor assay is reported in Renard et al., Nucleic Acids Res. (Feb. 15, 2001) 29:E21, which article describes a calorimetric assay that employs a substrate bound oligonucleotide which includes an NFKB consensus binding sequence, where both purified samples and cell extracts are assayed. In the assays reported in this document, only a single transcription factor, NFKB, is assayed.

There is continued interest in the development of new ELISA based assays for transcription factors and analogous DNA binding proteins. Of particular interest would be the development of such an assay that could be employed to detect multiple transcription factors in a single sample, including a cellular or nuclear extract, where the assay is more sensitive than EMSA.

Relevant Literature

Benotmane, et al. (1997) Analytical Biochemistry 250:181-185; Gubler et al., Biotechniques (1995) 18:1011-1014; Hibma et al., Nucleic Acids Res. (1994) 22: 3806-3807; McKay et al., Anal. Biochem (1998) 1:28:34; Mollo, Methods Mol. Biol. (2000) 130:235-246; Renard et al., Nuc. Acids Res. (Feb. 15, 2001) 29:E21; and Revzin, Biotechniques (1989) 7:346-355. See also: (a) U.S. Pat. Nos. 4,963,658; 4,978,608; 5,011,770; (b) WO 95/30026; WO 98/08096; WO 99/19510; WO 01/73115; and (c) EP 0 620 439 and EP 1 136 567.

### SUMMARY OF THE INVENTION

Methods for detecting, both qualitatively and quantitatively, the presence of at least one, usually a plurality of, DNA binding proteins, e.g., transcription factors, in a sample are provided. In the subject methods, a substrate having one or more DNA probes immobilized on a surface thereof, one for each DNA binding protein of interest, is contacted with a sample under conditions sufficient for binding complexes of the probes and their respective DNA binding proteins to be produced. In certain embodiments, the sample is a cellular or nuclear extract. Resultant binding complexes on the surface of the substrate are then detected and related to the presence of the DNA binding proteins of interest in the sample. Also provided are devices and systems for use in practicing the subject methods. The subject methods find use in a variety of different applications, e.g., the study of transcription factor profiles in response to a given stimulus, and the like.

### BRIEF DESCRIPTION OF THE FIGURES

FIG. 1 provides a table of various consensus sequences employed in a transcription factor assay according to the subject invention.

FIG. 2 provides a schematic view of a representative assay according to the subject invention. Double-stranded DNA immobilized on a 96-well plate captures the transcription factor from the nuclear extract. A transcription factor specific antibody detects the DNA-bound transcription factor. This complex is then quantified by the combination of a HRP conjugated antibody and its substrate.

FIG. 3. Sensitivity comparison of the TF-EIA and EMSA. (a) TF-EIA: Purified NFkB p50 protein in increasing concentrations (by a factor of two) from 0-25.6 μM was incubated with NFkB p50 wild-type dsDNA. EMSA: Purified NFkB p50 protein in increasing concentrations (by a factor of two) from 0-102.4 μM was incubated with ³²P-end-labeled NFkB p50 wild-type dsDNA. (b) Gel visualization of EMSA sigmoidal curve. Lane 13 is a supershift using anti-NFkB p50 polyclonal antibody incubated with ³² P-end-labeled NFkB p50 wild-type dsDNA and 12.8 μM of NFkB p50 purified protein which corresponds to the concentration used in lane 9.

FIG. 4. Dose response and competition assays for NFkB p50, ATF-2, and c-Fos. Dose response assays were performed by applying increasing amounts of nuclear extract (0-30 μg) to either wild-type or mutant dsDNA coated wells specific for each transcription factor. Meanwhile, in the competition assay, wild-type oligos or mutant oligos specific for each transcription factor in increasing concentration (25-200 ng) were incubated with 30 μg of nuclear extract and this mix was added to wild-type dsDNA coated wells. (a) NFkB p50 was detected by anti-NFkB p50 in HeLa stimulated with TNFα nuclear extract. (b) ATF-2 was detected by anti-ATF-2 in Jurkat nuclear extract. (c) c-Fos was detected by anti-c-Fos in HeLa stimulated with PMA nuclear extract. The data represents the means of three values ±SD.

FIG. 5. Antibody specificity of the TF-EIA for NFkB p50, ATF-2, and c-Fos. 30 μg of HeLa stimulated with TNFα nuclear extract was added to NFkB p50 wild-type dsDNA coated wells and incubated with anti-NFkB p50, anti-ATF-2, and anti-c-Fos. 30 μg of Jurkat nuclear extract was added to ATF-2 wild-type dsDNA coated wells and incubated with the same three antibodies. 30 μg of HeLa stimulated with PMA nuclear extract was added to c-Fos dsDNA coated wells and incubated with the same three antibodies. The data represents the means of three values ±SD.

FIG. 6. Multiple transcription factor profiling in different HeLa nuclear extracts. Three separate HeLa nuclear extracts, non-induced, induced with TNFα, and induced with PMA, were applied to wells coated with wild-type dsDNA corresponding to NFkB p50, NFkB p65, c-Rel, c-Fos, CREB-1 and ATF-2. This was followed by detection with the corresponding primary antibody. The data represents the means of two values ±SD.

FIG. 7. Multiple transcription factor profiling with Raji and NIH-3T3 cells. 30 μg of nuclear extracts from Raji cells and NIH-3T3 cells were prepared and added to the wells. Then the assay was developed as described. Mutant oligos did not show any binding events (data not show).

FIG. 8. Multiple trnascription factor profiling with Raji and U937 cells. 30 μg of nuclear extracts from Raji and U937 cells were used in each binding assay. The experiments were performed in triplicate.

FIG. 9 provides a diagram of the 48 DBP TransFactor Glass Array (format 3.0) employed in the experimental section, below.

FIG. 10. Comparison of single antibody versus mixing antibodies. Biotinylated wild type and mutant oligos for three different transcription factors were printed in each chamber on an eight-chamber slide. Nuclear extracts from Hela+TNFa (A), K562+PMA (B), Raji (C & D), U937 (E), or Jurkat (F) were incubated in respective chambers. For comparison, duplicated chambers were then incubated with either a single primary antibody NF-kBP50, or mixing antibodies of NF-kBp50, Oct-1, HSF-1 (A); single anti-EGR or mixing anti-EGR, NF-kBp65, Oct-2 (B); single anti c-Rel (C), anti-Max (D) or mixing anti-c-Rel, Max, p53 (C&D); single anti-SRF-1, or mixing anti-SRF-1, ATF2, pRb (E); or single anti-ATF2 or mixing anti-SRF-1, ATF2, pRb (F).

### DESCRIPTION OF THE SPECIFIC EMBODIMENTS

Methods for detecting the presence of at least one, usually a plurality of, DNA binding proteins (e.g., transcription factors) in a sample, both qualitatively and quantitatively, are provided. In the subject methods, a substrate having one or more DNA probes immobilized on a surface thereof, one for each DNA binding protein of interest, is contacted with a sample under conditions sufficient for binding complexes of the probes and their respective transcription factors to be produced. The sample may be purified DNA binding protein composition or a cellular/nuclear extract. Resultant binding complexes on the surface of the substrate are then detected and related to the presence of the DNA binding protein(s) (e.g., transcription factor(s)) of interest in the sample. Also provided are devices, kits and systems for use in practicing the subject methods. The subject methods find use in a variety of different applications, e.g., the study of transcription factor profiles in response to a given stimulus, screening for therapeutic agents, and the like. In further describing the subject invention, the subject methods will be described first, followed by a review of representative devices, systems and kits for use in practicing the subject methods.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and Devices

As summarized above, the subject invention is directed to methods of detecting the presence of one or more DNA binding proteins in a sample. In detecting the presence of the one or more DNA binding proteins in a sample, a given DNA binding protein can be detected either qualitatively or quantitatively. When the DNA binding protein of interest is detected qualitatively, the sample is typically screened to determine whether or not the DNA binding protein of interest is present in the sample. With quantitative detection, the concentration or amount of DNA binding protein of interest in the sample is determined. The quantitative detection may be relative or absolute, such that the amount of DNA binding protein of interest is determined relative to an arbitrary control value or is determined in absolute terms, e.g., mass/volume. In many embodiments, the presence of the DNA binding protein(s) of interest is determined quantitatively.

The DNA binding proteins of interest are proteins that specifically bind to a given, defined and specific stretch, domain or region of a DNA molecule, e.g., a known transcription factor binding sequence, a transcription factor consensus sequence, etc. In other words, the DNA binding proteins assayed by the subject methods are those that recognize a stretch of nucleotide residues in a DNA molecule, i.e., they specifically bind to a DNA recognition or consensus sequence. The recognition sequence may vary in length depending on the DNA binding molecule, but typically ranges from about 5 to about 25 bp, usually from about 6 to about 18 bp and more usually from about 6 to about 12 bp in length. The DNA binding proteins are typically proteins that specifically bind to double-stranded DNA. DNA binding proteins of interest include, but are not limited to: 1) regulator proteins, e.g., transcription factors; activators and co-activators, repressors and co-repressors; 2) proteins of the basal transcription complex, e.g. the holoenzyme and its mediator; 3) proteins that paticipate in DNA remodeling e.g. topoisomerase, helicase and ligase; 4) proteins involved in the structure and organization of chromatin e.g. histone, ATP-dependent remodeling cyclase, acetylase, Basic domain (bZIP, bHLH, bHLH-ZIP), Zinc finger domain (Cys2Cys2, Cys2His2, Cys6 clusters), Helix-turn-helix domain (homeo, winged helix, trp-clusters, TEA), beta-scaffold with minor groove contact (REL, MADS, TBP, HMG);and the like.

In many embodiments, the DNA binding protein(s) for which a sample is screened in the subject methods is a transcription factor. Transcription factors of interest include, but are not limited to: 1) Zinc finger (CH): EGR1, 2 and 3, SP1, YY1; 2) Zinc finger-zinc twist: RXR, HNF-4, ROR, PPARgamma, PR, ER, AR; 3) Zinc finger CC(G): GATA: 4) Zinc finger CC: YAF2: 5) Zinc finger C6 (yeast); 6) Homeodomain, homeobox protein: pbx-1a; 7) POU domain: Oct-1, pit; 8) BZIP: C/EBP, c-Fos, c-Jun, CREB: 9) Leucine zipper: GCF; 10) BHLH: Myo D; 11) HLH: Id1; 12) bHLH-Zip: cmyc; 13) HLH-zip: Vav; 14) MADS: MEF2-D; 15) HMG: TCF-4, LEF1: 16) Paired domain, paired box: Pax-5; 17). Paired domain, paired box+hemeodomain: Pax-7; 18) cold-shock domain: YB-1; 19) Rel: p50, p65, c-rel, NFAT; 20) Zinc finger+homeodomain: ATBF1: 21) Tip cluster: c-myb, IRF1: 22) Fork head domain: FOXO3-a, E2F-1; 23) TEA domain: TEF-1; 24) LIM domain+homeo domain: Lim-1; 25) HTH (no vertebrate); 26) Homeo, ZIP (no vertebrate); 27) Lim domain: Lmo 2; 28) Runt homology domain: AML1: 29) Histone fold: CPIA; 30) GCM: GCMa; 31) BHSH: AP-2alpha; 32) AP2 domain (no vertebrate); 33) Dof (no vertebrate); 34) WRKY domain (no vertebrate); 35) PHD finger (no vertebrate); 36) BED finger (no vertebrate); 37) T-box: Tbx 5; 38) Others that do not fall into a category: STATs, ATF-2, RB, p107, p53 and DP1; and the like.

Factors of interest include: AAF, abl, ADA2, ADA-NF1, AF-1, AFP1, AhR, AIIN3, ALL-1, alpha-CBF, alpha-CP1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, Barhl1, Barhl2, Barx1, Barx2, Bcl-3, BCL-6, BD73, beta-catenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, ChxlO, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, EIk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXM1a, FOXMlb, FOXM1c, FOXN1, FOXN2, FOXN3, FOX01a, FOX01b, FOXO2, FOXO3a, FOXO3b, FOXO4, FOXP1, FOXP3, Fra-1, Fra-2, FTF, FTS, G factor, G6 factor, GABP, GABP-alpha, GABP-betal, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCN5, GF1, GLI, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HEN1, HEN2, Hesxl, Hex, HIF-1, HIF-lalpha, HIF-1beta, HiNF-A, HiNF-B, HINF-C, HINF-D, HiNF-D3, HiNF-E, HiNF-P, HIP1, HIV-EP2, Hlf, HLTF, HLTF (Metl23), HLX, HMBP, HMG I, HMG I(Y), HMG Y, HMGI-C, HNF-1A, HNF-1B, HNF-1C, HNF-3, HNF-3alpha, HNF-3beta, HNF-3gamma, HNF4, HNF-4alpha, HNF4alpha1, HNF-4alpha2, HNF-4alpha3, HNF-4alpha4, HNF4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXB5, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1 H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, II-6 RF, INSAF, IPF1, IRF-1, IRF-2, irlB, IRX2a, Irx-3, lrx-4, ISGF-1, ISGF-3, ISGF3alpha, ISGF-3gamma, lsl-1, ITF, ITF-1, ITF-2, JRF, Jun, JunB, JunD, kappay factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHX5, LHX6.1a, LHX6.1b, LIT-1, Lmo1, Lmo2, LMX1A, LMX1B, L-My1 (long form), L-My1 (short form), L-My2, LSF, LXRalpha, LyF-1, LyI-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2D0B, MEF-2DA0, MEF-2DA′0, MEF-2DAB, MEF-2DA′B, Meis-1, Meis-2a, Meis-2b, Meis-2c, Meis-2d, Meis-2e, Meis3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF III-c, NF III-e, NF-1, NF-1A, NF-1B, NF-lX, NF-4FA, NF-4FB, NF-4FC, NF-A, NF-AB, NFAT-1, NF-AT3, NF-Atc, NF-Atp, NF-Atx, NfbetaA, NF-CLE0a, NF-CLE0b, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaB1, NF-kappaB1, precursor, NF-kappaB2, NF-kappaB2 (p49), NF-kappaB2 precursor, NF-kappaE1, NF-kappaE2, NF-kappaE3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrf1, Nrf-1, Nrf2, NRF-2betal, NRF-2gamma1, NRL, NRSF form 1, NRSF form 2, NTF, O2, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otx1, Otx2, OZF, p107, p130, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-1a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, Pm×2a, Pm×2b, PO-B, Pontin52, PPARalpha, PPARbeta, PPARgamma1, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (BJA-B), PU.1, PuF, Pur factor, R1, R2, RAR-alpha1, RAR-beta, RAR-beta2, RAR-gamma, RAR-gamma1, RBP60, RBP-Jkappa, Rel, RelA, RelB, RFX, RFX1, RFX2, RFX3, RFX5, RF-Y, RORalpha1, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP1b, SF-1, SHOX2a, SHOX2b, SHOXa, SHOXb, SHP, SIII-p110, SIII-p15, SIII-p18, SIM1, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1alpha, STAT1beta, STAT2, STAT3, STAT4, STAT6, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1,Tal-1beta, Tal-2, TAR factor, TBP, TBX1A, TBX1 B, TBX2, TBX4, TBX5 (long isoform), TBX5 (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbetal, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-MO15, TFIIH-p34, TFIIH-p44, TFIIH-p62, TFIIH-p80, TFIIH-p90, TFII-I, Tf-LF1, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-del2, WT1 -KTS, WT1-del2, X2BP, XBP-1, XW-V, XX, YAF2, YB-1, YEBP, YY1, ZEB, ZF1, ZF2, ZFX, ZHX1, ZIC2, ZID, ZNF174, etc.

For purposes of clarity and ease of description, the invention is now described further in terms of transcription factor detection. However, the nature of the DNA binding protein that is assayed by the present methods is not limited to transcription factors, as any type of DNA binding protein that specifically binds to a DNA recognition sequence may be assayed by the subject methods, as described above.

In practicing the subject methods, a fluid sample to be assayed is first contacted under DNA-protein binding conditions with a substrate having immobilized on a surface thereof a probe composition for each different transcription factor to be assayed. In other words, a probe composition specific for each different transcription factor to be detected in the sample is immobilized on the surface of the substrate. For example, if the substrate is to be used to detect the presence of just one transcription factor, then the substrate generally has a single probe composition immobilized on a surface. In yet other embodiments where the substrate is used in the detection of five different transcription factors, the substrate has five different probe compositions immobilized on its surface. The number of different or distinct probe compositions on the substrate surface may vary from one to a plurality, such that when a plurality of different probe compositions are present on the support surface, the number is at least about 2, usually at least about 5, where the number may be as high as about 10, 15, 25, 100, 200, 500, 1000 or higher. Any two given probe compositions are considered to be distinct or different if, under the assay conditions described below, they specifically bind to different transcription factors. Any two given transcription factors are considered to be distinct or different if they have a sequence identity that is less than about 95% as determined using MegAlign, DNAstar (1998) clustal algorithm as described in D. G. Higgins and P. M. Sharp, “Fast and Sensitive multiple Sequence Alignments on a Microcomputer,” (1989) CABIOS, 5: 151-153. (Parameters used are ktuple 1, gap penalty 3, window, 5 and diagonals saved 5).

The one or more probe compositions, described in greater detail below, are immobilized on a surface of a substrate. As such, the probe compositions are stably associated with the surface of a solid support, where the support may be a flexible or rigid support. By “stably associated” it is meant that the oligonucleotides of the spots maintain their position relative to the solid support (i.e., are immobilized on the support surface) under binding and washing conditions, described in greater detail below. As such, the probes that make up each probe composition can be non-covalently or covalently stably associated with the support surface based on technologies well known to those of skill in the art. Examples of non-covalent association include non-specific adsorption, binding based on electrostatic (e.g., ion, ion pair interactions), hydrophobic interactions, hydrogen bonding interactions, specific binding through a specific binding pair member covalently attached to the support surface (e.g., via biotin/streptavidin or neutravidin interaction), and the like. Examples of covalent binding include covalent bonds formed between the probe functionalities and a functional group present on the surface of the rigid support, e.g., —OH, NH₂, etc., where the functional group may be naturally occurring or present as a member of an introduced linking group.

As mentioned above, the probe compositions of the array are present on the surface of either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e., the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the probe compositions are presented or displayed may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.1 mm to 2 mm and more usually from about 0.2 to 1 mm. Thus, in one representative embodiment the support may have a micro-titre plate format, having dimensions of approximately 12×85 mm. In another representative embodiment, the support may be a standard microscope slide with dimensions of from about 25×75 mm.

In certain embodiments subject devices are substrates having a plurality of reaction chambers, where each reaction chamber includes a probe composition present on its bottom surface. By plurality is meant at least 2, usually at least 6, more usually at least 24, and most usually at least 96, where the number of different reaction chambers of the device may be as high as 384 or higher, but will usually not exceed about 450 and more usually will not exceed about 400. The overall size and configuration of the device will be one that provides for simple, manual handling, where the device may be disc shaped, slide shaped, and the like, where slide shaped (i.e., having a substantially rectangular cross-sectional shape, such as found in a microscope slide or a credit card) is preferred. The length of the device will typically range from 50 to 150 mm, usually from 70 to 130 mm and more usually from 75 to 128 mm, the height of the device will range from 2 to 20 mm, usually from 5 to 15 mm and more usually from 10 to 15 mm and the width of the device will range from 20 to 100 mm, usually from 20 to 90 mm and more usually from 25 to 87 mm.

In certain embodiments, each reaction chamber of the device will be a container having an open top, a bottom surface, and at least one wall surrounding the bottom planar surface in a manner sufficient to form a container, where the number of distinct walls surrounding the bottom surface will depend on the cross-sectional shape of the container, e.g. 1 wall for a container having a circular cross-sectional shape and 4 walls for a container having a square cross-sectional shape. The reaction chamber may have a variety of cross-sectional shapes, including circular, triangular, rectangular, square, pentagon, hexagon, etc., including irregular, but will usually have a rectangular or square cross-sectional shape. Therefore, the number of distinct walls surround the bottom planar surface of the reaction chamber will be at least one, and can be 2, 3, 4, 5, 6 or more, depending on the cross-sectional shape, but will usually be 4. In yet other embodiments, separate reaction chambers may be produced on a substrate by placing a barrier around different areas of a substrate, e.g., a rubber chamber, in order to produce the desired reaction chambers.

The area of the bottom surface will be sufficiently large to present at least one probe composition, and in certain embodiments two or more probe compositions, in a manner that makes the probes of the probe composition available for binding upon contact with the medium comprising the transcription factor(s) of interest. Generally, the area of the bottom surface will be at least about 9 mm², usually at least about 25 mm² and more usually at least about 30 mm², where the area may be as great as 8000 mm² or greater, but will usually not exceed 1300 mm² and more usually will not exceed 350 mm². The height of the walls will generally be uniform and will be sufficient to form a reaction chamber that is capable of holding a desired amount of fluid, e.g., reaction medium. As such, the height of each wall of the reaction chamber will be at least about 1 mm, usually at least about 3 mm and more usually at least about 5 mm and may be as high as 20 mm or higher, but will usually be no higher than about 15 mm, and more usually no higher than about 12 mm. The volume of fluid capable of being contained in the reaction chamber will generally range from about 5 μl to 75 ml, usually from about 10 μl to 3 ml and more usually from about 0.05 ml to 1.5 ml. Preferably, the walls will have a rectangular or square cross-sectional shape.

In yet other embodiments, regions of a planar substrate that are separated by hydrophobic strips, e.g., made hydrophobic by the presence of a hydrophobic film or coating of a hydrophobic material, or analogous structures serve as the reaction chambers described above. For an example of such embodiments, see Col. 11, line 42 through Col. 12, line 67 of U.S. Pat. No. 5,807,522, the disclosure of which is herein incorporated by reference.

In certain embodiments, a photolithographic process is employed to create a pattern of multiwells on a surface. Photo resists used can be positive and negative resists and can be applied by spraying, dipping, spin coating, plasma etching, vapor deposition or combinations of those. Surfaces may consist of glass, plastics, metals, minerals or combinations of those. Applications encompass manufacturing of multiwell plates with well sizes of a few Angstroms to 500 μm. Hydrophobic resists catch hydrophilic liquids on the well. As such, the height of the wells can be small. Basically any pattern can be manufactured. The method is easy and inexpensive. Resolution may be down to 80 nm depending on resist and “illumination” process. Advantages include ability to use small sample volumes, which feature is important in terms of miniaturization and throughput. The surface may be precoated streptavidin. The resist can be chosen of various existing compounds to keep the coated surface unchanged. Various photo resists exhibit a wide range of chemical and heat resistance. However, the resists can be simply dissolved in an appropriate solvent to wash them away from the resist coated surface if needed after performance of the assay (e.g. incubation) but before detection if needed, or after detection if needed for example if the surface needs to be reused for the same assay or a subsequent reaction/incubation. Multiple layers and coatings may be applied in that way to generate same, similar or different patterns than in the previous step. It is also possible to further modify the photo resist patterned surfaces with compounds other than photo resists (e.g. streptavidin) applying various techniques including plasma etching.

The substrates of the subject arrays may be fabricated from a variety of materials. The materials from which the substrate is fabricated should ideally exhibit a low level of non-specific binding during contact with the sample, as described below. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc. Also of interest are composite materials, such as glass or plastic coated with a membrane, e.g. nylon or nitrocellulose, etc.

The substrates of the subject device include at least one surface on which one or more probe compositions are present, where the surface may be smooth or substantially planar, or have irregularities, such as depressions or elevations. The surface on which the probe compositions are present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like.

Each probe composition is a collection of double stranded DNA molecules, where the collection is substantially homogenous such that there is substantially no variation in the molecules present in the composition. As such, all of the molecules have substantially the same length and substantially the same sequence, where any variation in terms of length does not exceed about 1 number % and usually does not exceed about 0.001 number %, while any two probe molecules in the composition have a sequence identity (as determined using the above described program) that is at least about 90%, usually at least about 95% and more usually at least about 99%. The length of the double stranded DNA probe molecules that make up the various probe compositions may vary depending on the specific transcription factor to which the probe is designed to bind, but typically is at least about 50 bp long, usually at least about 45 bp long and more usually at least about 40 bp long, where the length may be as long as 50 bp or longer, but generally does not exceed about 55 bp and usually does not exceed about 60 bp.

The sequence of the probes of the probe composition is chosen to provide for specific binding to the target transcription factor of interest. A variety of transcription factor recognition or consensus sequences are known in the art and may be used as probes in the devices of the present invention. Specific sequences of interest include those that specifically bind to: 1. Zinc finger (CH): EGR1,2 and 3, SP1, YY1. 2. Zinc finger-zinc twist: RXR, HNF-4, ROR, PPARgamma, PR, ER, AR. 3. Zinc finger CC(G): GATA. 4. Zinc finger CC: YAF2. 5. Zinc finger C6 (yeast). 7. Homeodomain, homeobox protein: pbx-1a. 8. POU domain: Oct-1, pit. 9. BZIP: C/EBP, c-Fos, c-Jun, CREB. 10. Leucine zipper: GCF. 11. BHLH: Myo D. 12. HLH: Id1. 13. bHLH-Zip: c-myc. 14. HLH-zip: Vav. 15. MADS: MEF2-D. 16. HMG: TCF-4, LEF1. 17. Paired domain, paired box: Pax-5. 18. Paired domain, paired box+hemeodomain: Pax-7.18. cold-shock domain: YB-1. 19. Rel: p50, p65, c-rel, NFAT. 20. Zinc finger+homeodomain: ATBF1. 21. Tip cluster: c-myb, IRF1. 22. Fork head domain: FOXO3-a, E2F-1. 23. TEA domain: TEF-1. 24. LIM domain+homeo domain: Lim-1. 25. HTH no vertebrate). 26. Homeo, ZIP (no vertebrate). 27. Lim domain: Lmo 2. 28. Runt homology domain: AML1. 29. Histone fold: CPIA. 30. GCM: GCMa. 31. BHSH: AP-2alpha. 32. AP2 domain (no vertebrate). 33. Dof (no vertebrate). 34. WRKY domain (no vertebrate). 35. PHD finger (no vertebrate) 36. BED finger (no vertebrate). 37. T-box: Tbx 5. 38. Others that do not fall into a category: STATs, ATF-2, RB, p107, p53 and DP1; and the like.

Factors of interest include: MF, abl, ADA2, ADA-NF1, AF-1, AFP1, AhR, AIIN3, ALL-1, alpha-CBF, alpha-CP1, alpha-CP2a, alpha-CP2b, alphaH0, alphaH2-alphaH3, Alx-4, aMEF-2, AML1, AML1a, AML1b, AML1c, AML1DeltaN, AML2, AML3, AML3a, AML3b, AMY-1 L, A-Myb, ANF, AP-1, AP-2alphaA, AP-2alphaB, AP-2beta, AP-2gamma, AP-3 (1), AP-3 (2), AP4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, BarhIl, Barhl2, Barxl, Barx2, Bcl-3, BCL-6, BD73, betacatenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCMT-binding factor, CCAAT-binding factor, CCF, CCG1, CCK-1a, CCK-1b, CD28RC, cdk2, cdk9, Cdx-1, CDX2, Cdx4, CFF, ChxlO, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, DIx-1, Dlx-2, Dlx-3, Dlx-4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, EIk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVIl, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXXM1a, FOXXM1b, FOXXM1c, FOXN1, FOXN2, FOXN3, FOXO1a, FOXO1b, FOXO2, FOXO3a, FOXO3b, FOXO4, FOXP1, FOXP3, Fra-1, Fra-2, FTF, FTS, G factor, G6 factor, GABP, GABP-alpha, GABP-betal, GABP-beta2, GADD 153, GAF, gammaCAAT, gammaCAC1, gammaCAC2, GATA-1, GATA-2, GATA-3, GATA-4, GATA-5, GATA-6, Gbx-1, Gbx-2, GCF, GCMa, GCN5, GF1, GLI, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1B, HEF-1T, HEF-4C, HEN1, HEN2, Hesx1, Hex, HIF-1, HIF-lalpha, HIF-1beta, HiNF-A, HINF-B, HiNF-C, HINF-D, HiNF-D3, HiNF-E, HINF-P, HIP1, HIV-EP2, Hlf, HLTF, HLTF (Met123), HLX, HMBP, HMG I, HMG I(Y), HMG Y, HMGI-C, HNF-1A, HNF-1B, HNF-1C, HNF-3, HNF-3alpha, HNF-3beta, HNF-3gamma, HNF-4, HNF-4alpha, HNF-4alpha1, HNF-4alpha2, HNF-4alpha3, HNF-4alpha4, HNF-4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB1, HOXB13, HOXB2, HOXB3, HOXB4, HOXB5, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1H′, Id2, Id3, Id3/Heir-1, IF1, IgPE-1, IgPE-2, IgPE-3, IkappaB, IkappaB-alpha, IkappaB-beta, IkappaBR, 11-1 RF, IL-6 RE-BP, 11-6 RF, INSAF, IPF1, IRF-1, IRF-2, irlB, IRX2a, lrx-3, Irx-4, ISGF-1, ISGF-3, ISGF3alpha, ISGF-3gamma, IsI-1, ITF, ITF-1, ITF-2, JRF, Jun, JunB, JunD, kappaY factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHX5, LHX6.1a, LHX6.1b, LIT-1, Lmo1, Lmo2, LMX1A, LMX1B, L-My1(1ongform), L-My1(shortform), L-My2, LSF, LXRalpha, LyF-1, Lyl-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 M form), MEF-2C (465 M form), MEF-2C (473 AA form), MEF-2C/delta32 (441 AA form), MEF-2D00, MEF-2DOB, MEF-2DA0, MEF-2DA′0, MEF-2DAB, MEF-2DA′B, Meis-1, Meis-2a, Meis-2b, Meis-2c, Meis-2d, Meis-2e, Meis-3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF III-c, NF III-e, NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, NfbetaA, NF-CLE0a, NF-CLE0b, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NFkappaB(-like), NF-kappaB1, NF-kappaB1 precursor, NF-kappaB2, NF-kappaB2 (p49), NF-kappaB2 precursor, NF-kappaE1, NF-kappaE2, NF-kappaE3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrf1, Nrf-1, Nrf2, NRF-2betal, NRF-2gamma1, NRL, NRSF form 1, NRSF form 2, NTF, O2, OCA-B, Oct-1, Oct-2, Oct-2.1, Oct-2B, Oct-2C, Oct4A, Oct-4B, Oct-5, Oct-6, Octa-factor, octamer-binding factor, oct-B2, oct-B3, Otx1, Otx2, OZF, p107, p130, p28 modulator, p300, p38erg, p45, p49erg, p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx1-a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, Pm×2a, Pm×2b, PO-B, Pontin52, PPARalpha, PPARbeta, PPARgamma1, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (BJA-B), PU.1, PuF, Purfactor, R1, R2, RAR-alpha1, RAR-beta, RAR-beta2, RAR-gamma, RAR-gamma1, RBP60, RBP-Jkappa, Rel, Re1A, RelB, RFX, RFX1, RFX2, RFX3, RFX5, RF-Y, RORalpha1, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP-1b, SF-1, SHOX2a, SHOX2b, SHOXa, SHOXb, SHP, SIII-p110, SIII-p15, SIII-p18, SIM1, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1 alpha, STAT1 beta, STAT2, STAT3, STAT4, STAT6, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1,Tal-1beta, Tal-2, TAR factor, TBP, TBX1A, TBX1 B, TBX2, TBX4, TBX5 (long isoform), TBX5 (short isoform), TCF, TCF-1, TCF-lA, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbeta1, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-MO15, TFIIH-p34, TFIIH-p44, TFIIH-p62, TFIIH-p80, TFIIH-p90, TFII-I, Tf-LF1, TfLF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1TREF2, TRF (2), TTF-1, TxRE BP, TxREF, UBF, UBP-1, UEF-1, UEF-2, UEF-3, UEF-4, USFI, USF2, USF2b, Vav, Vax-2, VDR, vHNF-1A, vHNF-1B, vHNF-1C, VITF, WSTF, WT1, WT1I, WT1I-KTS, WT1 I-del2, WT1-KTS, WT1-del2, X2BP, XBP-1, XW-V, XX, YAF2, YB-1, YEBP, YY1, ZEB, ZF1, ZF2, ZFX, ZHX1, ZIC2, ZID, ZNF174, etc.

In certain embodiments, the probe sequences may be mutant oligos, in which the point mutation of one or more, e.g., one or two, nucleotides of the DNA-protein binding sites in a consensus sequence is present. The mutant oligo can be used to differentiate the specific DNA-protein binding and non-specific binding.

The amount of probes that make up a probe composition may vary, but generally is at least about 10 ng, usually at least about 50 ng and more usually at least about 100 ng, where the amount may be as high as 10 μg or higher, but typically does not exceed about 1 μg and usually does not exceed about 0.1 μg. The area of the substrate surface that is covered by a given probe composition may vary, but is generally at least about 0.1 to 1 cm², usually from about 0.1 to 0.5 cm².

As indicated above, where two or more different probe compositions are present on an array, each may be present in its own reaction chamber, such that a fluid barrier separates any two probe compositions on the array and each probe composition is isolated fluidically from any other probe composition on the array. In yet other embodiments, two or more probe compositions of the array are not separated by a fluidic barrier. For example, all of the probe compositions may be present on a planar substrate surface, e.g., glass surface, where there is no barrier between any two compositions on the surface. Alternatively, groups of compositions may be isolated from each other. In certain embodiments where a plurality of different reaction chambers are present on an array, and each reaction chamber includes two or more different oligo probes for different transcription factors, probes placed together in any given reaction chamber are selected that do not cross-react with the transcription factors of the other probes in the same reaction chamber.

The above devices can be fabricated using any convenient protocol. One convenient protocol is provided in the Experimental Section, below. However, other protocols are also possible with the primary consideration being one of conveniences.

The sample that is contacted with the substrate surface may vary greatly, depending upon nature of the assay to be performed. In general, the sample is an aqueous fluid sample. The amount of fluid sample also varies with respect to the nature of the device, the nature of the sample, etc. In many embodiments, the amount of sample that is contacted with the substrate surface ranges from about 2.5 μg to 100 μg, usually from about 5 μg to 50 μg and more usually from about 5 μg to 30 μg.

In many embodiments, the fluid sample is naturally occurring sample, where the sample may or may not be modified prior to contact with the substrate. In many embodiments, the fluid sample is obtained from a physiological source, where the physiological source is typically eukaryotic, With physiological sources of interest including sources derived from single celled organisms such as yeast and multicellular organisms, including plants and animals, particularly mammals, where the physiological sources from multicellular organisms may be derived from particular organs or tissues of the multicellular organism, or from isolated cells or cellular compartments, e.g., nucleus, cytoplasm, etc., derived therefrom. In obtaining the fluid sample, the initial physiological source (e.g., tissue) may be subjected to a number of different processing steps, where such processing steps might include tissue homogenation, nucleic acid extraction and the like, where such processing steps are known to the those of skill in the art. Of particular interest in many embodiments is the use of cellular extracts and nuclear extracts as the fluid sample. Representative methods of preparing both cellular and nuclear extracts are provided in the Experimental Section, below.

While the subject methods are highly sensitive and are able to detect very small amounts of the target transcription factor, the concentration of the target transcription factor in the sample is generally at least about 0.3 μM, usually at least about 0.5 μM and more usually at least about 1 μM, where the concentration may be as high as 5 μM or higher, but generally does not exceed about 10 μM and usually does not exceed about 30 μM.

In certain embodiments, the sample is an aqueous fluid sample of one or more purified transcription factors, which may be isolated from a naturally occurring source or recombinantly produced. Such fluid samples of purified transcription factor protein find use in a variety of applications, e.g., screening assays to identify agents that modulate the binding of a transcription factor to its recognition sequence, such as agonists or antagonists of the transcription factor of interest. Fluid samples of purified transcription factor may have a single transcription factor or a plurality of different transcription factors, where when a plurality is present, the number is at least about 2, usually at least about 5 and may be as high as 10 or higher, e.g., 15, 25, 50, 75, 100, or higher. In such samples, the identity, and often amount, of the transcription factors, as well as other components present in the sample are typically known. The amount of each transcription factor in the sample typically ranges from about 0.75 ng to 100 ng, usually from about 2 ng to 40 ng and more usually from about 2 ng to 20 ng. In addition to water and the transcription factor(s), the samples in this embodiment may also include a number of additional components, e.g., buffers, ions, chelating agents, etc. A representative binding buffer is disclosed in the experimental section, below.

With either the cellular/nuclear extracts or purified transcription factor samples described above, the sample may include a blocking agent for reducing non-specific binding interactions. Blocking agents of interest include, but are not limited to: nonfat milk, BSA, gelatin, preimmune serum and the like, as is known in the art.

As summarized above, the first step in the subject methods is to contact the substrate surface displaying the one or more probe compositions with the fluid sample under conditions sufficient for specific binding between any transcription factor present in the sample and its recognized DNA sequence displayed on the substrate surface to occur. Contact may occur using any convenient protocol. As such, the sample may be applied to the substrate surface, placed in the reaction chambers of the substrate surface, flowed across the substrate surface, or the substrate surface may be immersed in the fluid sample, etc.

Following contact, contact between the surface and fluid sample is maintained for a period of time sufficient for binding between transcription factors in the sample and their recognized probes on the substrate surface to occur. As such, the substrate surface and the sample are incubated for a period of time and under conditions sufficient for binding between probes and their corresponding transcription factors in the sample to occur. The sample and substrate are typically incubated for a period of time ranging from about 5 min to 2 hours, usually from about 15 min to 2 hours and more usually from about 30 min to 1 hour. The temperature during this incubation period generally ranges from about 0 to about 37° C. usually from about 15 to 30° C. and more usually from about 18 to 25° C. Where desired, the substrate and sample may be agitated during incubation, e.g., by shaking, stirring, etc.

Following incubation, transcription factor/probe complexes present on the surface of the substrate are detected. In many embodiments and depending on the labeling scheme employed, non-bound transcription factors are removed from the substrate surface prior to detection. Where non-bound transcription factors are removed prior to detection of surface bound complexes, the non-bound transcription factors may be conveniently removed by washing or other suitable protocol. Washing typically involves contacting the surface with a wash fluid followed by removal of the fluid from the surface, e.g., by flushing the surface with a wash fluid. A number of different wash fluids/wash protocols are known in the art that are suitable for use with array applications, and such may be employed in the present invention.

Any convenient detection protocol may be employed to detect the presence, and often amount, of surface bound complex. In many embodiments, a signal producing system is employed to detect the presence of surface bound complexes. By signal producing system is meant a system of one or more reagents that work to provide a detectable signal that can be related to the presence of surface bound complex. In many embodiments, the presence of surface bound complexes is detected by employing labeled affinity reagents, e.g., antibodies or binding fragments or mimetics thereof, e.g., Fv, F(ab′)₂, scFv, and Fab, etc., specific for the transcription factor portion of the surface bound complexes to be detected. As such, in many embodiments, the signal producing system that is employed is an antibody based or affinity reagent based signal producing system.

Detecting may occur using one or more different signal producing system fluid compositions that each include one or more reagent members of a signal producing system. For example, where each probe composition is present on the substrate in its own fluidically isolated region, e.g., a well of a microtitre plate, a different fluid composition for each probe composition may be employed, where the different fluid compositions differ from each other in terms of the specificity of signal producing system, e.g., in terms of labeled affinity reagent specificity, and each fluid composition employed includes only a single type of affinity reagent. In alternative embodiments, a fluid composition that includes a plurality of different labeled affinity reagents may be employed, e.g., in those situations where two or more different probe compositions are not fluidically isolated from each other. For example, in embodiments where five different probe compositions are present on a planar surface and not isolated from each other, detection may be achieved by employing a fluid composition of five different detection antibodies (e.g., an antibody cocktail), one for each of the different compositions.

Antibodies/fragments thereof that find use in the detection of surface bound complexes are those that specifically bind to the transcription factor of interest, and more specifically to a position that is available when the transcription factor is bound to its corresponding probe, e.g., an epitope that is still accessible following binding of the transcription factor to its probe. The antibodies or binding fragments thereof may be obtained from a commercial source or prepared de novo, using antibody generation protocols well known to those of skill in the art, e.g., monoclonal antibody generation technology, polyclonal antibody generation technology, phage display, etc.

As mentioned above, the antibody or binding fragment thereof is labeled to provide for detection of the surface bound complex to which it binds. A variety of protein labeling schemes are known in the art and may be employed, the particular scheme and label chosen being the one most convenient for the particular assay protocol being performed. As such, the label may be a directly detectable or indirectly detectable label. A variety of different labels may be employed, where such labels include fluorescent labels, isotopic labels, enzymatic labels, particulate labels, etc. For example, suitable labels that provide for direct detection include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), cyanine dyes, e.g. Cy5, Cy3, BODIPY dyes, e.g. BODIPY 630/650, Alexa542, etc. Suitable isotopic labels include radioactive labels, e.g. ³²P, ³³P, ₃₅S, ³H. Other suitable labels include size particles that possess light scattering, fluorescent properties or contain entrapped multiple fluorophores. Examples of labels which permit indirect measurement of the presence of the antibody include enzymes where a substrate may provided for a colored or fluorescent product. For example, the antibodies may be labeled with a covalently bound enzyme capable of providing a detectable product signal after addition of suitable substrate. Instead of covalently binding the enzyme to the antibody, the antibody may be modified to comprise a first member of specific binding pair which specifically binds with a second member of the specific binding pair that in conjugated to the enzyme, e.g., the antibody may be covalently bound to biotin and the enzyme conjugate to streptavidin.

In antibody based signal producing systems, a single antibody may be employed or two or more different antibodies working in concert may be employed. For example, a single antibody may be employed that includes both a transcription factor specific binding region and a directly or indirectly detectable label. Alternatively first and second antibodies may be employed, where the first antibody is specific for the transcription factor and the second antibody is directly or indirectly detectable and binds to the first antibody, e.g., the second antibody is labeled anti-lgG. In yet other embodiments, three or more antibodies are employed that work in concert in a manner analogous to the above described two antibody system.

In many embodiments, an ELISA signal producing system is employed to detect the presence of surface bound complexes. ELISA signal producing systems are well known to those of skill in the art of immunoassays. See e.g., Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; and Ishikawa, et al., (eds.) Enzyme Immunoassay, Kgaku Shoin, Tokyo, 1981.

In such assays, the surface bound complex is first labeled with a suitable enzyme, e.g., with a single antibody conjugate or two or more antibodies that work in concert to ultimately label the surface complex with the enzyme. Enzymes finding use include, but are not limited to, malate dehydrogenase, staphylococcal nuclease, delta5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

Following labeling with the enzyme, and typically after one or more washing steps as described above, the enzyme is reacted with an appropriate substrate, preferably a chromogenic substrate, in such a manner as to produce a chemical moiety which can be detected, for example, by spectrophotometric, fluorimetric or by visual means. The detection can be accomplished by colorimetric methods which employ a chromogenic substrate for the enzyme, where suitable substrates include, but are not limited to: o-phenylenediamine (OPD), 3,3′,5,5′-tetramethylbenzidine (TMB), 3,3′-diaminobenzide tetrahydrochloride (DAB), and the like. In this step, a fluid composition of the substrate, e.g., an aqueous preparation of the substrate, is typically incubated with the substrate surface for a period of time sufficient for the detectable product to be produced. Incubation typically lasts for a period of time ranging from about 10 sec to 2 hours, usually from about 30 sec to 1 hour and more usually from about 5 min to 15 min at a temperature ranging from about 0 to 37° C., usually from about 15 to 30° C. and more usually from about 18 to 25° C.

In yet other two part systems, the second antibody is labeled with a directly detectable label, e.g., a fluorescent or isotopic label, as described above.

At the end of the incubation period, the product is detected and related to the presence of the complex on the substrate surface. Detection may also be accomplished by visual comparison of the extent of enzymatic reaction of a substrate in comparison with similarly prepared standards. Detection may be accomplished using any convenient protocol and device, including but not limited to: spectrophotometric, fluorimetric or by visual means.

The final step is to relate the detected signal generated from the detectably labeled surface bound complexes to the presence of the corresponding transcription factor(s) in the fluid sample that has been assayed. The detected signal can be used to qualitatively determine whether or not the transcription factor of interest is present in the sample that has been assayed. Alternatively, the detected signal can be used to quantitatively determine the amount of the transcription factor of interest in the assayed sample. Quantitative determination is generally made by comparing a parameter of the detected signal, e.g., intensity, with a reference value (such as the intensity of signal generated from a known amount of label).

As such, the above process can be used to detect the presence of one or more transcription factors in a fluid sample, either quantitatively or qualitatively.

A feature of the subject invention is that the subject methods are more sensitive than EMSA. Particularly, the subject methods are more sensitive than a corresponding EMSA control, as disclosed in the experimental section below. The increase in sensitivity is at least about 5-fold, usually at least about 10-fold.

Utility

The above methods and devices find use in a variety of different applications in which one desires to detect the presence of one or more transcription factors in a fluid sample. Because of the nature of the subject methods and devices, they are particularly suited for use in applications in which a plurality of different transcription factors are assayed simultaneously, e.g., they are particularly suited for use high throughput applications where one wishes to detect the presence of two or more transcription factors simultaneously, such as applications where one wishes to detect at least 5, 10, 15, 25, or more transcription factors simultaneously.

One particular application in which the subject invention finds use in profiling the transcription factor population of a tissue, cell or subcellular location, i.e., in transcription factor profiling of a tissue, cell or portion of a cell (a subcellular location). By “transcription factor profiling” is meant that the amount of one or more, usually a plurality of, e.g., 2, 5, 10, 15, 25 or more, different transcription factors in the cell or component thereof, e.g., nucleus, is determined to obtain information on the nature of the various transcription factors that are present and affecting the cell or compartment thereof. The transcription factor profile can be obtained and compared to the transcription factor profile of one or more different types of cells, so as to obtain comparative data with respect to the nature of the cell being assayed. Alternatively, the transcription profile can be detected before and after a cell is subjected to a given stimulus, so as to identify information regarding how a cell responds to a given stimulus. In yet other embodiments, one can monitor changes in the transcription profile of a cell over time, so as to elucidate how development changes a cell. As such, the subject invention finds use in profiling transcription factor families, signal transduction pathways, the detection of novel transcription factors and the like.

The subject invention also finds use in all applications where EMSA is employed, including, but not limited to: all protein/DNA interaction assays in which EMSA currently finds use.

Yet another application in which the subject invention finds use is in high throughput screening for agents that modulate the binding activity of transcription factor to its recognized DNA sequence, e.g., in screening for transcription factor agonists and antagonists in a high throughput manner. In such assays, a sample of a plurality of transcription factors for which the identification of a modulatory agent is desired, e.g., such as sample of two or more purified transcription factors, as described above, is contacted with a device as described above in the presence of a candidate agent and the effect of the candidate agent on the binding of the transcription factor to its oligonuceotide probe is determined, e.g., by reference to a control. The observed effect or lack thereof is then related to the modulatory capacity of the candidate compound. In this manner, a given agent can be screened for modulatory activity with respect to more than one transcription factor simultaneously. For example, a potential candidate inhibitory agent can be screened simultaneously against a plurality of different transcription factors by contacting a sample containing the transcription factors of interest with a device having a probe for each transcription factor of interest in the presence of the candidate agent and observing the effect of the candidate agent on the binding of each of the transcription factors of interest to its respective probe.

Kits and Systems

As summarized above, also provided are kits and systems for use in practicing the subject methods. The kits and systems at least include the subject high throughput devices, as described above. The kits and systems may also include a number of optional components that find use in the subject methods. Optional components of interest include a signal producing system or components thereof, e.g., an antibody based signal producing system or components thereof, including but not limited to: antibodies specific for transcription factors of interest, antibody enzyme conjugates, chromogenic substrates, etc. The signal producing system may be in the form of one or more distinct signal producing system fluid compositions, where each fluid composition may include one or more, including a plurality of, different affinity reagents, e.g., labeled antibodies, such that the fluid composition may contain a single antibody or be an antibody cocktail. Finally, in many embodiments of the subject kits, the kits will further include instructions for practicing the subject methods or means for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

The following examples are offered by way of illustration and not by way of limitation.

### Experimental

I.

A. Materials and Methods

1. Oligonucleotides

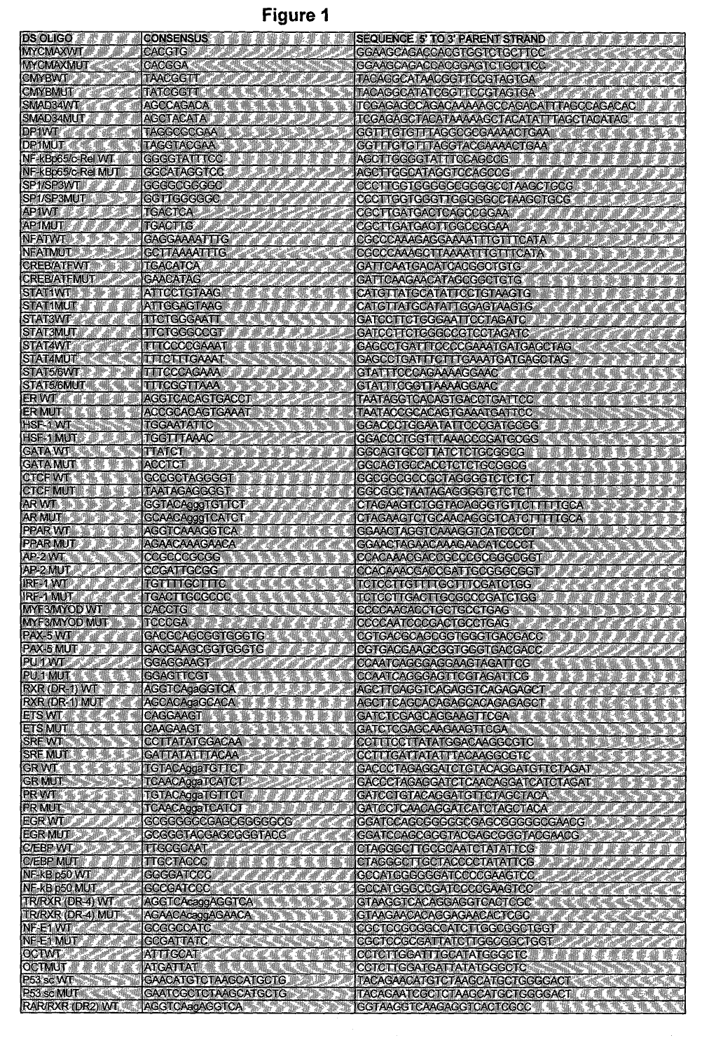

The parent and complementary single stranded oligonucleotides, corresponding to the wild-type and mutated transcription factor consensus sequences (FIG. 1), were purchased from OPERON (Alameda, Calif.). Each oligonucleotide was HPLC purified, and the parent strand was biotinylated at the 5′ end by OPERON. Before use, both strands were annealed by heating at 100° C. in TE buffer for 5 minutes and gradually cooled to room temperature.

2. Nuclear Extract and Purified Protein

Nuclear extracts were made using the TransFactor Extraction Kit (Clontech, Palo Alto, Calif. #K2064-1). Cells were grown to 80-90% confluency and were either non-induced, HeLa and Jurkat, or induced, HeLa. For induction HeLa cells were incubated with either 0.1 μg/ml TNF-α (Clontech, 8157-1) for 30 minutes, or 2 μg/ml PMA (Sigma, St. Louis, Mo.) for 2 hours. Cells were harvested, washed, and pelleted. The cells were resuspended in 5 volumes of hypotonic lysis buffer, dependent on the pelleted cell volume, and incubated on ice for 15 minutes. The cells were then centrifuged, resuspended in 2 volumes of hypotonic lysis buffer based on the original pelleted cell volume and disrupted with a syringe. The disrupted cells were centrifuged to isolate the cytoplasmic fraction, which was removed and stored at−70° C. The pellet containing the nuclei was resuspended in extraction buffer, disrupted with a syringe and gently shaken for 30 minutes at 4° C. Finally, the material was centrifuged for 5 minutes at high speed and the supernatant corresponding to the nuclear extract was removed and stored at −70° C. Purified human recombinant NFkB p50 was purchased from Promega (Madison, Wis.).

3. Transcription Factor Enzyme-Linked Immunoassay (TF-EIA)

Neutravidin coated 96-well strip plates (Pierce, Rockford, Ill.) were incubated with 100 μl per well of 33 nM biotinylated double-stranded DNA (dsDNA), corresponding to related wild-type and mutated consensus sequences, in TransFactor buffer (Clontech) for 1 hour at room temperature. After each step 3 washes were performed. Each well was then blocked with 3% nonfat milk in TransFactor buffer for 1 hour. Nuclear extract or purified transcription factor diluted in TransFactor buffer plus 3% nonfat milk were added at a volume of 50μl per well and incubated for 1 hour at room temperature. Primary antibody diluted in TransFactor buffer plus 3% milk (Table 1) was then added at 100 μl per well and incubated at room temperature for 1 hour. After washing, 100 μl per well of secondary antibody diluted in TransFactor buffer with 3% milk (1:1000) was added and incubated at room temperature for 30 minutes. Finally, after addition of 100 μl per well of TMB (3,3,5,5-tetramethylbenzidine) substrate (Bio-Rad, Hercules, Calif.), color development was detected at 655 nm with BIO-RAD Model 550 microplate reader.

4. Antibodies

The primary antibodies used include: anti-NFkB p50 (Upstate Biotech, Waltham, Mass. pAb #06886), anti-NFkB p65 (Upstate Biotech, Santa Cruz, Calif., pAb #06418), anti-c-Rel (Santa Cruz Biotech, pAb #SC-71), anti-c-Fos (Santa Cruz Biotech., pAb #SC7201), anti-CREB-1 (Santa Cruz Biotech., pAb #SC186), and antiATF-2 (Santa Cruz Biotech., pAb #SC187). The secondary antibody used was: antirabbit IgG-HRP (BD-Transduction Labs, #R14745),

5. Oligonucleotide Competition Assay

Wells were incubated with wild type dsDNA oligonucleotides in TransFactor buffer for 1 hour at room temperature. Increasing amounts (25 ng to 200 ng) of wild type or mutant competition oligotucleotides were added to 30 μg of Nuclear Extract in 50 μl total volume of TransFactor buffer plus 3% milk. After blocking, this mixture was then added to each well coated with the wild type dsDNA for 1 hour. The remaining steps of the TF-EIA were then performed as previously described.

6. Electrophoretic Mobility Shift Assay (EMSA)

Double-stranded DNA oligonucleotide (wild-type) was labeled with ³²P using a 3′-end labeling kit (Amersham Pharmacia Biotech, Piscataway, N.J.). Nuclear extract or purified transcription factor were incubated with 2.5 μl of the ³²P-oligonucleotide probe for 20 minutes in 20 mM HEPES, pH 7.9, 40 mM KCI, 1 mM MgCl₂, 100 μM EDTA, 500 μM dithiothreitol, 6% glycerol, and 0.1 mg/ml poly (dl-dC). The samples were then fractionated on 0.5× TBE (100 mM Tris borate, pH 8, 2 mM EDTA), 5% acrylamide gel. For supershift analysis, 0.5 μl of polyclonal antibody or 1 μl of (1 mg/ml) monoclonal antibody was incubated with ³²P-labeled DNA-transcription factor complex.

B. Results and Discussion

1. Principle of the Transcription Factor Enzyme-Linked Immunoassay (TF-EIA)

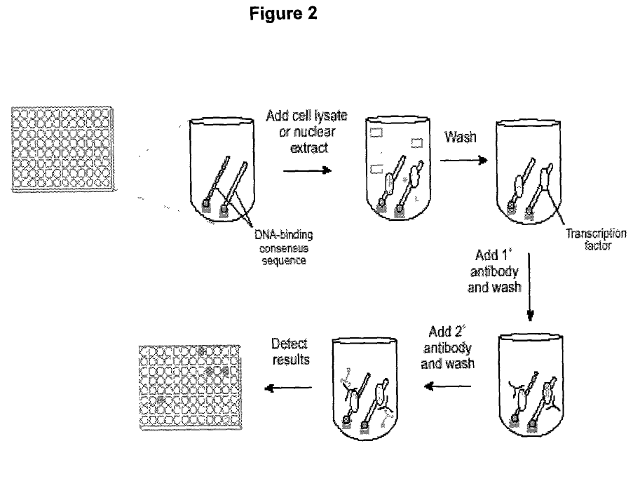

Wild-type and mutated dsDNA consensus sequences for each transcription factor were immobilized on neutravidin-coated 96-well plates. The DNA coated wells were then incubated with purified protein, mammalian nuclear extract, or mammalian cellular extract. DNA-transcription factor complexes were detected with primary antibodies specific for the target transcription factors and a horseradish peroxidase (HRP) conjugated secondary antibody. Finally the TMB substrate was added to the wells and color development was measured with a microplate reader (FIG. 2)

2. Increased Sensitivity of the TF-EIA Compared with the EMSA

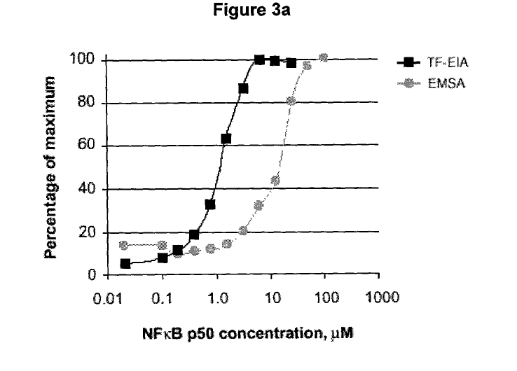

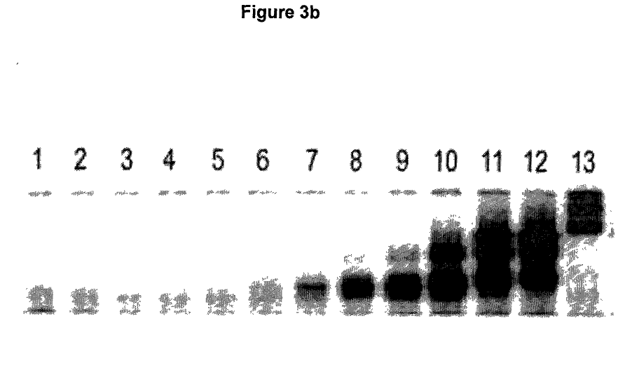

The sensitivities of the TF-EIA and EMSA to detect DNA-protein binding activity were compared using purified recombinant human NFkB p50 protein. Neutravidin-coated wells were incubated with wild-type NFkB p50 dsDNA (FIG. 1), at the saturating concentration of 33 nM. The dsDNA was then exposed to purified NFkB p50 protein at concentrations in the range of 0 μM to 25.6 μM. Anti-NFkB p50 antibodies detected NFkB p50 protein that bound to the dsDNA. The result was a sigmoidal curve with the saturation plateau at 6 μM (FIG. 3a). We defined the lowest detection point to be the concentration corresponding to two times the background absorbance, which was 0.3 M of purified NFkB p50 protein. ³²P-end-labeled NFkB p50 wild-type dsDNA was incubated with increasing amounts of purified NFkB p50 protein from 0 μM to 102.4 μM. The free and protein-bound dsDNA was separated by gel electrophoresis and the optical density of the bands were determined on the phosphorimager (FIG. 3b). The lowest detected concentration based on two times the background, replacing absorbance with optical density of the band, was approximately 3 μM. From this point the band intensity gradually increased until it reached a saturation point at approximately 100 μM. At high concentrations of purified protein, increasing amounts of multimeric forms were seen. These bands were included with the lower band in the determination of optical density in order to compare with the TF-EIA which has no way to differentiate protein-DNA binding forms such as monomers or oligomers.

As seen in FIG. 3a, after normalization both the TF-EIA and the EMSA showed similar sigmoidal curves. However, based on the lowest detected protein concentration of both assays, the TF-EIA (0.3 μM) exhibited a 10-fold higher sensitivity than the EMSA (3 μM).

3. Analysis of Transcription Factor-DNA Binding Activity in Mammalian Nuclear Extract

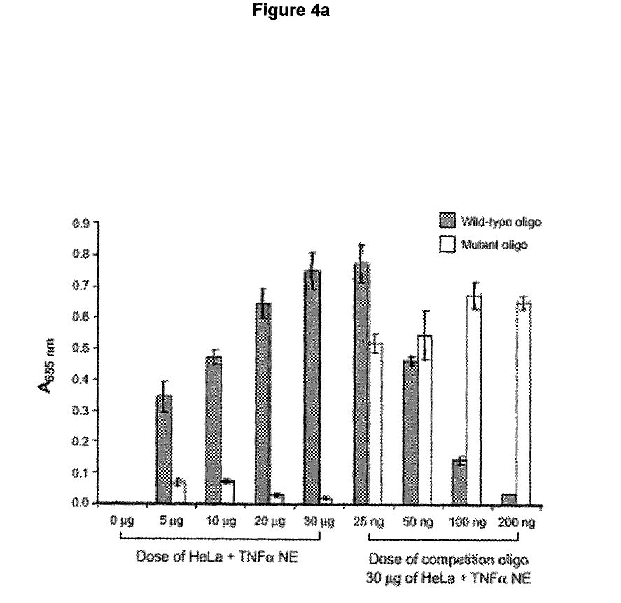

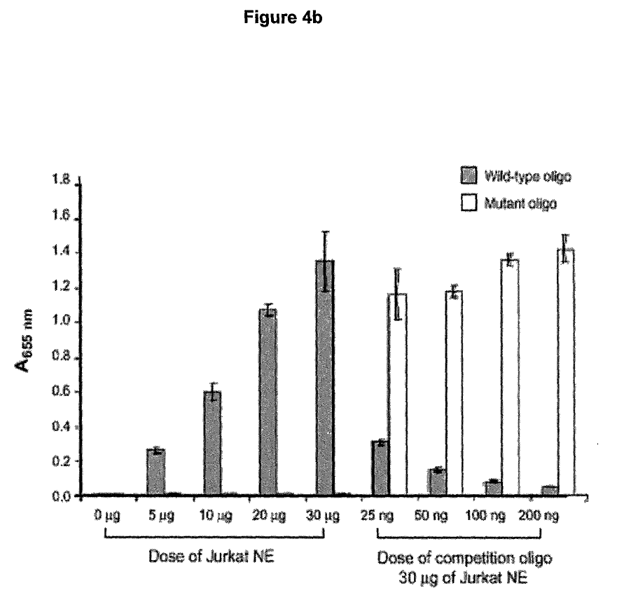

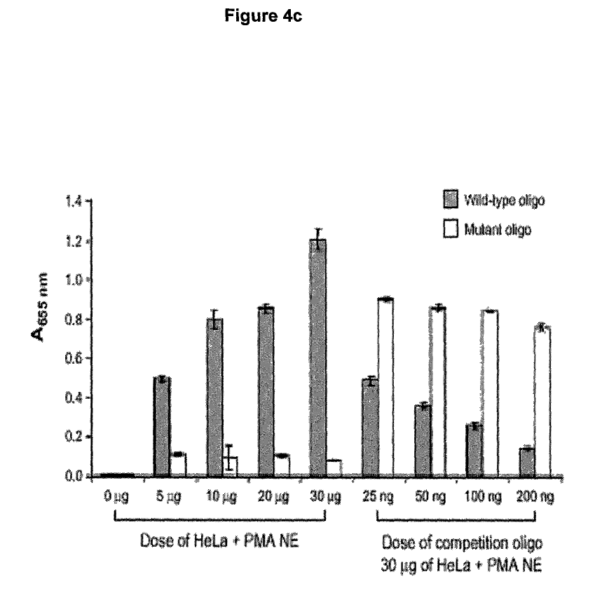

In many cases purified protein is either unavailable or difficult to purify, so we tested the ability of the TF-EIA to detect specific transcription factor-DNA binding using nuclear extract of mammalian cells. NFkB p50 wild-type and mutant dsDNA consensus sequences (FIG. 1) were incubated with increasing amounts of nuclear extract (0-30 μg) from HeLa cells induced with TNFα (FIG. 4a). The protein binding to wild-type dsDNA increased proportionally with the amount of nuclear extract applied. Meanwhile, no increase in binding was observed in the wells coated with mutant dsDNA.

Typically, an oligo competition assay is performed in the EMSA to assess binding specificity and to determine the key bases in the protein-binding DNA consensus sequence. The same competition assay was performed in the TF-EIA. Non-biotinylated oligos corresponding to the wild-type or mutant NFkB p50 consensus sequences were mixed with 30 μg of HeLa induced with TNFα nuclear extract (the highest dose amount) and this was then added to wells coated with the biotinylated wild-type NFkB p50 dsDNA (FIG. 4a). With the addition of increasing amounts of wild-type competitor oligo we observed a gradual decrease of DNA-protein binding activity, while no corresponding decrease was seen with the addition of increasing amounts of mutant oligos.

We performed the dose and competition assay with members of two other transcription factor families, ATF-2 (FIG. 4b) and c-Fos (FIG. 4c), using Jurkat nuclear extract and HeLa induced with PMA nuclear extract, respectively. The three transcription factors we tested all resulted in similar dose responses and DNA-protein binding was successfully competed away with the addition of a specific wild-type oligo. In each case DNA-protein binding was detected at the lowest dose of 5 μg of nuclear extract and the detectable binding was almost completely abolished with the addition of 40 times competitor oligo. In order to quantify the amount of transcription factor present in the nuclear extract, purified protein could be used to produce a standard curve. Slightly different detection sensitivities were observed for each transcription factor. These differences may be caused by the following; each nuclear extract may contain different concentrations of each transcription factor, or, alternately, the binding affinities of the transcription factor for the consensus sequence and the binding affinities of the antibodies for proteins could be different.

4. Specificity of Antibody-Transcription Factor Binding

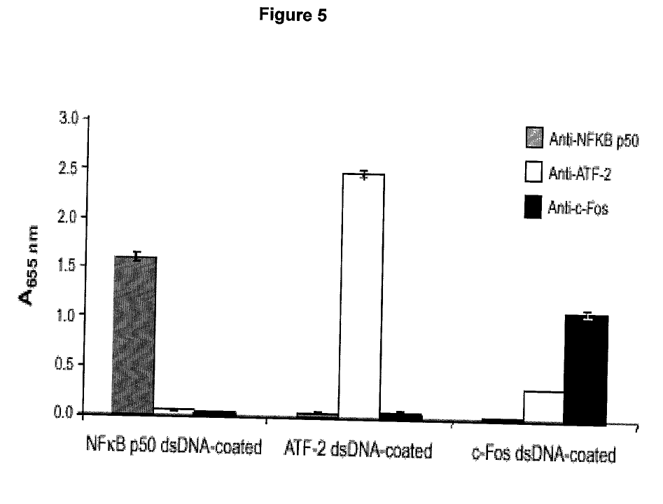

In order to verify that each antibody is bound to its specific transcription factor the TF-EIA was performed using specific and non-specific antibodies. NFkB p50 wild-type dsDNA coated wells were incubated with 30 μg of HeLa induced with TNFα nuclear extract and one of three antibodies: anti-NFkB p50, anti-ATF-2, and anti-cFos (FIG. 5). The NFkB p50 protein-DNA complex was detected by anti-NFkB p50, but not by anti-ATF-2 or anti-c-Fos. Similarly, only the specific antibody detected ATF-2 protein-DNA complexes and c-Fos protein-DNA complexes when using Jurkat nuclear extract and HeLa induced with PMA nuclear extract, respectively. No antibody cross-reactivity was observed in this assay which indicates that positive signals usually correspond to the specific dsDNA-protein-antibody complexes. However, protein-protein interactions among transcription factors naturally occur, such as the NFkB p50 zand NFkB p65 heterodimer. In this assay, we were able to separately detect both NFkB p65 and NFkB p50 using their respective antibodies on a plate coated only with NFkB p50 wild-type dsDNA (data not shown).

5. Profiling the DNA Binding Activity of Multiple Transcription Factors in Different Nuclear Extracts

It is often of interest to compare transcription factor activation in induced versus non-induced cells. The activation levels of six transcription factors involved in immunodisease were profiled simultaneously in three different HeLa nuclear extracts. Nuclear extract from HeLa, Hela induced with TNFα, and HeLa induced with PMA were added to wells containing wild-type or mutant dsDNA corresponding to NFkB p50, NFkB p65, c-Rel, c-Fos, CREB-1, and ATF-2 (FIG. 1). In all cases, the mutant dsDNA coated wells resulted in little or no signal (data not shown).

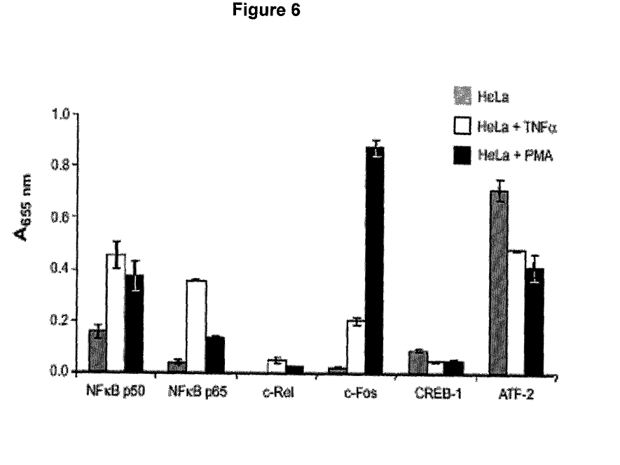

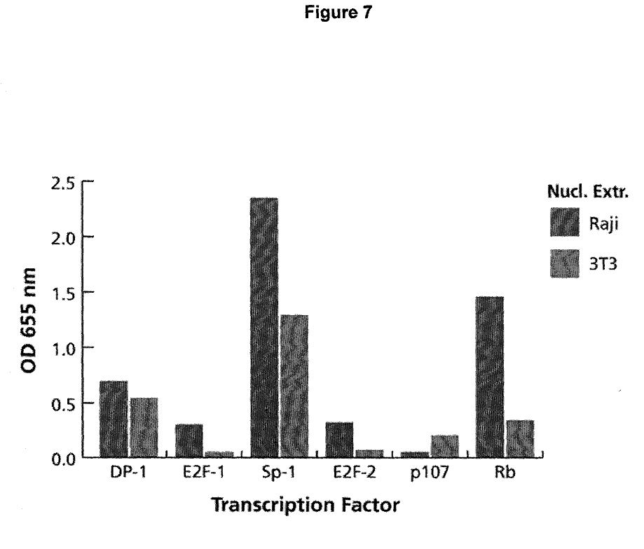

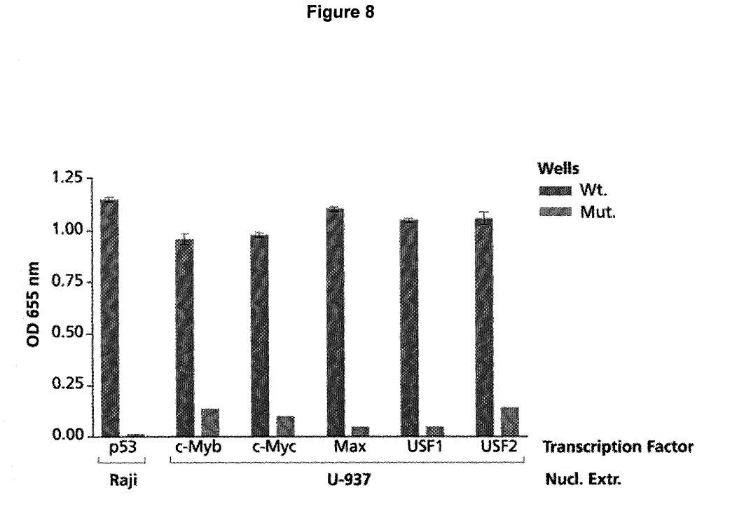

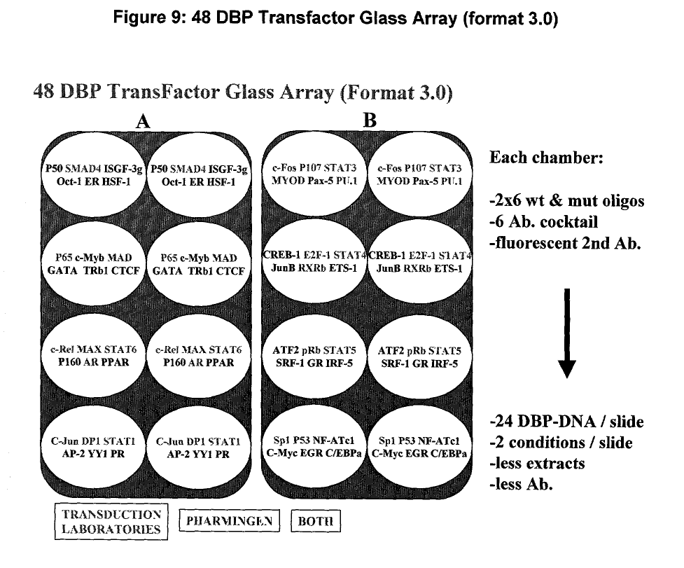

The transcription factor NFkB is normally sequestered in the cytosol due to its association with IkB. Upon stimulation this association is dissolved and NFkB translocates to the nucleus. Increased levels of both NFkB p50 and NFkB p65 were detected in induced HeLa nuclear extract when compared to non-induced HeLa nuclear extract (FIG. 6).

Activation of the protein kinase C signaling pathway by PMA causes c-Fos to translocate to the nucleus. A significant increase in c-Fos binding activity in HeLa induced with PMA nuclear extract was observed when compared with non-induced HeLa nuclear extract (FIG. 6).

In this profiling experiment we looked at a specific type of nuclear extract stimulated with different conditions to focus on transcription factors involved in inflammation. Although c-Rel and CREB-1 were not activated in these nuclear extracts (FIG. 6) we saw high levels of endogenous c-Rel in non-induced Raji nuclear extract and high levels of CREB-1 in non-induced PC-12 nuclear extract (data not shown). The ATF-2 protein is ubiquitous and continually expressed, thus ATF-2 exhibits high endogenous levels in all the HeLa nuclear extracts (FIG. 6), along with non-induced Raji, PC-12, and Jurkat nuclear extracts (data not shown).

We have developed an alternative to the EMSA based on the ELISA platform. Compared to the EMSA the TF-EIA has ten fold higher sensitivity than the EMSA, takes a short time to run, and uses no radioactivity. Due to the sensitivity of the TF-EIA, it can be used to study DNA-protein binding events in nuclear extract, whole cell extract, and may be effective with tissue extract. Also, quantitative studies can be performed by using purified protein as a standard. Like the EMSA, the TF-EIA can be used for competition studies or to detect novel transcription factors. In the case of the TF-EIA, when no antibody is available, the putative transcription factor can be fused with a tagged expression vector and detected with a tag-specific antibody (data not shown).

The TF-EIA contains an inherent flexibility to screen for activation or inhibition of DNA binding protein activity in a high throughput format, and is easily expandable to 384 wells. This could be especially useful in drug discovery and cancer research. In addition, with the completion of the human genome project more and more transcription factors will be identified, thus an even higher capacity platform will be required. The TF-EIA can be adapted to the array format by immobilization of the dsDNA on glass and the use of an antibody cocktail. The miniaturization resulting from the microarray will require less sample and antibody to be used and accelerate the ability to analyze more transcription factor-DNA binding events simultaneously.

II. ELISA-Based Protein-DNA Binding Assay on Glass Array.

A. First Assay

Six μM DNA oligos for the wild type or mutant consensus sequences corresponding to a number of different transcription factors were biotin-labeled and printed on the streptavidin coated slides. The slides were incubated for one hour in blocking solution (20 mM Hepes, pH 7.6, 50 mM KCI, 10 mM (NH₄)₂S0₄, 1 mM DTT and EDTA, 0.2% Tween-20, 3% dry milk), then incubated with 1 nM, 10 nM purified NF-kB, or 3 μg, 30 μg nuclear extract from PMA (2 μg/ml) treated Hela cells. The incubation of purified protein or nuclear extract was performed in blocking solution for one hour, followed by three washes with blocking solution. The slides were then incubated for one hour in a primary antibody cocktail including antibodies against all the transcription factors presented on the array. Three washes with blocking solution were applied and followed by one half hour incubation with a Cy3-conjugated secondary antibody (Amersham Pharmacia Biotech anti-mouse 1:200 dilution, antirabbit 1:500 dilution) cocktail. The slides were then washed four times with wash buffer (blocking solution without dry milk), which were examined with fluorescence slide reader. The whole procedure was performed at room temperature.

Purified NF-kB p50 specifically binds to wild type NF-kB p50 and NF-kB p65 oligos, as they share the same consensus sequences. Some non-specific binding was also detected. Increased levels of non-specific binding was observed with 10 nM NF-kB p50 when compared with 1 nM purified protein was used in the experiment. Three μg Hela cells treated with PMA nuclear extract did not give any binding signal, while specific protein DNA binding to wild type NF-kB p50, NF-kB p65, and c-Jun consensus sequences were detected when 30 μg nuclear extract was used in the experiment. Some non-specific binding was also detected.

B. ELISA-Based Protein-DNA Binding Assay on Glass Array.

Six μM DNA oligos for the wild type or mutant consensus sequences corresponding to 24 or 48 of different transcription factors were biotin-labeled and printed on the streptavidin coated slides into 8 different chambers as described. The slides were incubated for one hour in blocking solution (20 mM Hepes, pH 7.6, 50 mM KCI, 10 mM (NH₄)₂S0₄, 1 mM DTT and EDTA, 0.2% Tween-20, 3% dry milk), then incubated with 1M purified protein (c-Jun, NF-kBP50), or 0.12 μg/μl nuclear extract from treated or untreated cells. The incubation of purified protein or nuclear extract was performed in blocking solution for one hour, followed by three washes with blocking solution. The slides were then incubated for one hour in a primary antibody cocktail including antibodies against all the transcription factors present in each chamber or one primary antibody against one transcription factor present in the chamber. Three washes with blocking solution were applied and followed by half hour incubation with a Cy3-conjugated secondary antibody (Amersham Pharmacia Biotech anti-mouse 1:200 dilution, anti-rabbit 1:500 dilution) cocktail. The slides were then washed four times with wash buffer (blocking solution without dry milk), which were examined with fluorescence slide reader. The whole procedure was performed at room temperature.

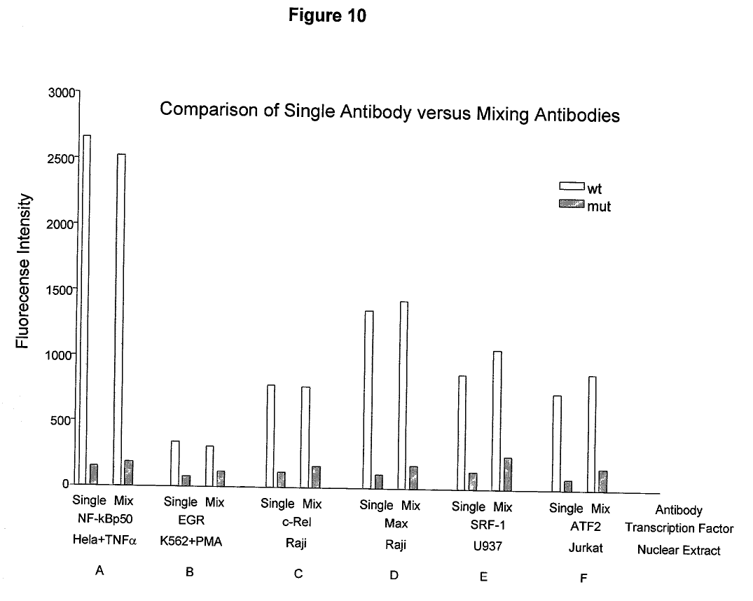

Specific protein DNA binding to the wild type oligos was detected when the nuclear extract with activated transcription factor was used for the incubation. No binding was detected for the mutant oligos. The binding to one or more transcription factors was shown depending on one or cocktail of primary antibody was added, and the transcription factors being activated in the specific nuclear extract. To compare binding specificity, a side by side comparison using either a single primary antibody or an antibody cocktail including all the transcription factors in each chamber was performed. The results (FIG. 10) showed that when same nuclear extract was used in the experiment, there was no significant difference of the protein binding to the specific wild type oligos when either a single or a cocktail of primary antibody was used in the experiment. Yet when a cocktail of antibodies was added in a chamber, more than one transcription factor-DNA binding could be shown, such as in Raji nuclear extract, both c-Rel and Max showed positive signals (FIGS. 10C & 10D).

It is evident from the above results and discussion that the subject invention provides many improvements over currently employed assays for assaying DNA/protein binding interactions, such as EMSA. Advantages of the subject invention include high sensitivity, ability to work with impure samples, e.g., cell or nuclear extracts, adaptability to a high throughput format, and the like. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for detecting the presence of a DNA binding protein in a sample, said method comprising:
(a) contacting a substrate having a probe specifically bound by a DNA binding protein immobilized on a surface thereof with said sample;
(b) detecting any binding complexes of said probe to the DNA binding protein to obtain assay data; and
(c) relating said assay data to the presence of the DNA binding protein in the sample;
wherein said method is characterized by having a sensitivity that is greater than the sensitivity of an EMSA control assay.

2. The method according to claim 1, wherein said DNA binding protein is a transcription factor.

3. The method according to claim 1, wherein said sample is a purified sample of the transcription factor.

4. The method according to claim 1, wherein said sample is a cellular extract.

5. The method according to claim 4, wherein said cellular extract is a nuclear extract.

6. The method according to claim 1, wherein said DNA binding protein is a transcription factor and said surface includes at least two different probes for at least two different transcription factors, wherein said method is a method of assaying for the presence of at least two different transcription factors simultaneously.

7. The method according to claim 6, wherein said substrate includes at least five different probes for at least 5 different transcription factors.

8. The method according to claim 2, wherein said binding complexes are detected using a signal producing system that includes an affinity reagent specific for said transcription factor.

9. The method according to claim 8, wherein said affinity reagent is directly detectable.

10. The method according to claim 8, wherein said affinity reagent is indirectly detectable.

11. The method according to claim 2, wherein said method quantifies the amount of the transcription factor in the sample.

12. The method according to claim 2, wherein said probe comprises a consensus sequence for the transcription factor.

13. The method according to claim 2, wherein said surface includes at least two different probes for at least two different transcription factors, wherein said at least two different probes are not separated from each other by a fluid barrier and said method is a method of assaying for the presence of at least two different transcription factors simultaneously.

14. The method according to claim 13, wherein said binding complexes are detected using a signal producing system that includes an affinity reagent specific for each of said at least two different transcription factors present in a single signal producing system fluid composition.

15. A method for quantitating the amount of two or more different transcription factors in a sample, said method comprising:
(a) contacting said sample with a substrate having immobilized on a surface thereof a distinct probe composition for each of said two or more different transcription factors, wherein each of the distinct probe compositions is made up of a double-stranded nucleic acid molecule that includes a transcription factor consensus sequence;
(b) detecting any binding complexes of the probes and transcription factors to obtain assay data;
(c) relating said assay data to the amount of said at least two transcription factors in the sample

16. The method according to claim 15, wherein said sample is a cellular extract.

17. The method according to claim 16, wherein said sample is a nuclear extract.

18. The method according to claim 15, wherein a fluidic barrier separates any two distinct probe compositions on said surface.

19. The method according to claim 18, wherein said substrate is a multiwell plate and each distinct probe composition is present in a separate well of the multiwell plate.

20. The method according to claim 15, wherein a fluid barrier does not separate at least two distinct probe compositions on said surface.

21. The method according to claim 15, wherein said method is a method for quantitating at least five different transcription factors in a sample.

22. The method according to claim 21, wherein said detecting step comprises using a signal producing system that includes an affinity reagent specific for a transcription factor.

23. The method according to claim 22, wherein said affinity reagent is directly detectable.

24. The method according to claim 22, wherein said affinity reagent is indirectly detectable.

25. An assay device for detecting at least five different transcription factors in a sample, wherein said device comprises:
a substrate having at least five different probe compositions immobilized on a surface thereof, wherein each of the at least five different probe compositions is made up of a double-stranded nucleic acid molecule that includes a transcription factor consensus sequence.

26. The assay device according to claim 25, wherein said device includes at least 10 different probe compositions.

27. The assay device according to claim 25, wherein at least two of said at least five different probe compositions are not separated by a fluid barrier.

28. The assay device according to claim 25, wherein any two probe compositions on said array are separated by a fluid barrier.

30. A kit for quantitating the presence of at least five different transcription factors in a sample, said kit comprising:
(a) a substrate having at least five different probe compositions immobilized on a surface thereof, wherein each of the at least five different probe compositions is made up of a double-stranded nucleic acid molecule that includes a transcription factor consensus sequence; and
(b) a signal producing system for each of the at least five different transcription factors.

31. The kit according to claim 30, wherein said signal producing system comprises an affinity reagent specific for a transcription factor.

32. The kit according to claim 31, wherein said affinity reagent is directly detectable.

33. The kit according to claim 31, wherein said affinity reagent is indirectly detectable.

34. The kit according to claim 33, wherein said signal producing system further comprises a second affinity reagent specific for the indirectly detectable affinity reagent.

35. The kit according to claim 34, wherein said second affinity reagent includes an enzyme moiety that converts a substrate into a chromogenic product.

36. The kit according to claim 31, wherein said signal producing system is a single fluid composition that includes a different affinity reagent for each of said at least 5 different transcription factors.

37. A system for quantitating the presence of at least five different transcription factors in a sample, said system comprising:
(a) a substrate having at least five different probe compositions immobilized on a surface thereof, wherein each of the at least five different probe composition is made up of a double-stranded nucleic acid molecule comprising a transcription factor consensus sequence; and
(b) a signal producing system for each of the at least five different transcription factors.

38. The system according to claim 37, wherein said signal producing system includes an affinity reagent specific for a transcription factor.

39. The system according to claim 38, wherein said affinity reagent is directly detectable.

40. The system according to claim 38, wherein said affinity reagent is indirectly detectable.

41. The system according to claim 40, wherein said signal producing system includes a second affinity reagent specific for said indirectly detectable affinity reagent.

42. The system according to claim 41, wherein said second affinity reagent includes an enzyme moiety that converts a substrate into a chromogenic product.
